(19) United States
(12) Patent Application Publication
(10) Pub. No.: US 2026/0248397 A1
(43) Pub. Date: Aug. 27, 2026

(54) METHOD AND APPARATUS FOR NONINVASIVE BLOOD PRESSURE ESTIMATION USING FOOTWEAR-INTEGRATED PRESSURE SENSING

(71) Applicant: VIDICOR INC, Laguna Beach, CA (US)

(72) Inventor: Alexander Holland, Laguna Beach, CA (US)

(21) Appl. No.: 19/546,202

(22) Filed: Feb. 20, 2026

Related U.S. Application Data
(60) Provisional application No. 63/761,953, filed on Feb. 22, 2025.
(60) Provisional application No. 63/979,830, filed on Feb. 10, 2026.
(60) Provisional application No. 63/982,764, filed on Feb. 13, 2026.

(51) Int. Cl. A61B 5/022; A61B 5/00; A61B 5/0205; A61B 5/021; A61B 5/024; A61B 5/0535; A61B 5/0537; A61B 5/08; A61B 5/107; A61B 5/20; A61B 90/00; G01G 19/44

(52) U.S. Cl. CPC A61B 5/02225; A61B 5/0205; A61B 5/02108; A61B 5/02141; A61B 5/02405; A61B 5/02444; A61B 5/0535; A61B 5/0537; A61B 5/0816; A61B 5/107; A61B 5/201; A61B 5/4035; A61B 5/4833; A61B 5/4878; A61B 5/6807; A61B 5/681; A61B 5/7203; A61B 5/7246; A61B 5/725; A61B 5/726; A61B 5/7275; A61B 5/742; A61B 5/746; A61B 90/06; G01G 19/44; A61B 2090/064; A61B 2560/0223; A61B 2562/0247; A61B 2562/046; A61B 2562/168

(57) ABSTRACT

Methods and apparatuses for noninvasive, continuous blood pressure estimation using one or more pressure sensors integrated into footwear, such as slippers, shoes, insoles, or padded socks. The sensors measure time-varying plantar pressure signals that include both weight-bearing and cardiovascular components. When a wearer stands or walks, the body weight load compresses underlying plantar arteries against the sensor surface, creating an oscillometric interaction between externally applied mechanical pressure and internal arterial blood pressure. Signal processing extracts blood-pressure-induced pulsatile modulations from the measured plantar pressure signal and estimates systolic blood pressure, diastolic blood pressure, and mean arterial pressure by analyzing the envelope of these modulations as a function of the applied weight-bearing pressure. The system operates passively and continuously without an inflatable cuff, without requiring user intervention, and without interrupting the wearer's daily activities.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims the benefit and priority of U.S. Provisional Application No. 63/761,953 filed Feb. 22, 2025, and titled “FootSens: A Smart Shoe Platform,” of U.S. Provisional Application No. 63/979,830, filed Feb. 10, 2026, and titled “FootSens: A Smart Footwear Platform,” and of U.S. Provisional Application No. 63/982,764 filed Feb. 13, 2026 and titled “FootSens: A Smart Footwear Platform,” the entire content of each of which is hereby incorporated by reference and forms a part of this specification for all purposes.

### BACKGROUND

### Field

The present disclosure relates to methods and apparatus for noninvasive blood pressure measurement. More specifically, it relates to estimating arterial blood pressure from plantar pressure signals acquired by sensors embedded in footwear, where the wearer's body weight provides a naturally varying compression of plantar arteries analogous to the externally applied pressure of an oscillometric arm cuff instrument.

### Description of the Related Art

Blood pressure is among the most important cardiovascular vital signs. Sustained elevation of blood pressure, known as hypertension, is a leading risk factor for heart disease, stroke, kidney disease, and other serious conditions. Despite its clinical importance, blood pressure is frequently measured too infrequently to capture its natural variability. The reasons are primarily practical: conventional measurement methods are cumbersome, uncomfortable, or require deliberate effort by the user.

The most widely used home blood pressure instrument employs an inflatable arm cuff connected to an automated oscillometric monitor. The cuff is wrapped around the upper arm, inflated to temporarily occlude the brachial artery, and then slowly deflated while a pressure sensor inside the cuff detects small pulsatile oscillations superimposed on the decaying cuff pressure. These oscillations arise from the interaction between the externally applied cuff pressure and the internal arterial blood pressure. The oscillometric envelope, namely the amplitude of the pulsatile oscillations as a function of the applied cuff pressure, reaches a maximum at or near the mean arterial pressure (MAP). Systolic blood pressure (SBP) and diastolic blood pressure (DBP) are estimated from characteristic ratios of the envelope amplitude relative to its peak.

Although oscillometric arm cuff instruments are sufficiently accurate for clinical use, they are inconvenient for frequent or continuous monitoring. Each measurement requires the user to sit quietly, apply the cuff, initiate inflation, and wait for the measurement cycle to complete. As a result, most individuals, even those with diagnosed hypertension, obtain only sporadic measurements that may not adequately represent their true blood pressure variability over the course of a day.

Continuous finger cuff devices (e.g., volume-clamp or vascular unloading methods) can provide beat-to-beat blood pressure waveforms but require the wearer to have a finger cuff and associated pneumatic apparatus connected during monitoring, which is impractical for routine home use and incompatible with normal daily activities.

Wrist-worn or watch-based devices have been proposed that estimate blood pressure indirectly from photoplethysmography (PPG) pulse transit time, pulse wave analysis, or other surrogate signals. However, the accuracy of these methods has generally not been sufficient for medical decision-making without frequent recalibration against a reference standard, and the correlation between PPG-derived surrogates and true arterial blood pressure can be confounded by many physiological and environmental factors.

There remains a need for a blood pressure monitoring method that is (a) passive and continuous, requiring no deliberate user action; (b) accurate enough to support medical decision-making; (c) compatible with normal daily activities such as standing and walking; and (d) inexpensive and comfortable enough for routine home use.

### SUMMARY

The present disclosure provides a method and apparatus for noninvasive, continuous estimation of arterial blood pressure using one or more pressure sensors integrated into footwear worn during normal daily activities and in some optional embodiments using PPG or electrical impedance sensors integrated into the footwear.

In one aspect, the disclosed technology provides a blood pressure sensing apparatus including: (a) one or more pressure sensors disposed within footwear, and capable of measuring a time-varying plantar pressure signal from a foot of a wearer including a weight-bearing component and a cardiovascular pulsatile component, the cardiovascular pulsatile component arising from interaction between the wearer's body weight and arterial blood pressure in plantar vasculature; (b) signal processing circuitry coupled to the one or more pressure sensors and capable of digitizing the plantar pressure signal; and (c) one or more processors that can: (i) separate or identify cardiovascular pulsatile components within the plantar pressure signal, (ii) determine the amplitude of the pulsatile components as a function of the applied weight-bearing component, and (iii) estimate at least one blood pressure parameter from a relationship between the pulsatile amplitude and the weight-bearing pressure magnitude; where the apparatus estimates blood pressure without an inflatable cuff and without requiring deliberate user action beyond wearing the footwear.

In some embodiments, the at least one blood pressure parameter includes mean arterial pressure estimated as the weight-bearing pressure at which the pulsatile amplitude is maximized. In further embodiments, the one or more processors can estimate systolic blood pressure and diastolic blood pressure from weight-bearing pressures at which the pulsatile amplitude falls to characteristic fractions of a peak amplitude. In some embodiments, the relationship between the pulsatile amplitude and the weight-bearing pressure magnitude forms an oscillometric envelope analogous to an oscillometric envelope produced by an inflatable arm cuff instrument. In some embodiments, separating or identifying the cardiovascular pulsatile component includes applying at least one of: a high-pass filter, an adaptive filter using inertial measurement data as a reference, empirical mode decomposition, or wavelet decomposition.

In some embodiments, the one or more processors can estimate the weight-bearing component by low-pass filtering the plantar pressure signal. In some embodiments, the one or more processors can estimate the at least one blood pressure parameter using a machine learning model trained on simultaneously acquired plantar pressure data and reference blood pressure measurements. In further embodiments, the machine learning model is trained on population-wide data and is fine-tuned for an individual wearer using one or more reference blood pressure measurements. In further embodiments, the machine learning model includes at least one of: a neural network, a recurrent neural network, a transformer, a convolutional neural network, a gradient-boosted decision tree, or a Gaussian process.

In some embodiments, the one or more pressure sensors include flexible capacitive force transducers having flexible electrodes separated by a compressible dielectric. In further embodiments, the flexible capacitive force transducers include a multilayer electrode-dielectric stack with vertically interdigitated electrode and dielectric layers. In further embodiments, the compressible dielectric exhibits an effective compressive stiffness that increases with applied pressure.

In some embodiments, the footwear includes a sealed, flexible enclosure filled with a viscous liquid, and at least one of the one or more pressure sensors is mounted within or on the enclosure in hydraulic communication with the viscous liquid so as to measure hydrostatic pressure reflecting an aggregate plantar pressure applied by the foot. In further embodiments, the viscous liquid includes at least one of: glycerine, silicone oil, mineral oil, or a biocompatible gel. In further embodiments, the viscous liquid provides mechanical low-pass filtering that attenuates high-frequency gait impact artifacts while preserving the cardiovascular pulsatile component. In further embodiments, the viscous liquid reduces mechanical impedance mismatch between the foot and the pressure sensor, thereby improving coupling of arterial blood pressure pulsations into the measured pressure signal. In further embodiments, a single pressure sensor within the sealed enclosure senses an area-averaged plantar pressure over a contact area of the foot by hydrostatic pressure equilibration through the viscous liquid.

In some embodiments, at least one pressure sensor is positioned to overlie or be adjacent to a lateral plantar artery of the wearer's foot. In some embodiments, the one or more pressure sensors are distributed across multiple regions of an insole of the footwear, including at least two of: a heel region, a metatarsal region, a medial arch region, and a lateral arch region. In some embodiments, the one or more processors can estimate heart rate from a frequency of the cardiovascular pulsatile component. In some embodiments, the one or more processors can estimate respiration rate from a modulation of the cardiovascular pulsatile component or of the weight-bearing component.

In some embodiments, the apparatus includes a communication interface that can receive photoplethysmography (PPG) data from an external wearable device, and the one or more processors can: (a) calculate a blood pressure estimate based at least in part on both the plantar-pressure-based blood pressure estimate and the PPG-derived blood pressure estimate when both the footwear and the external wearable device are worn simultaneously, or (b) monitor blood pressure, based at least in part on the PPG-derived blood pressure estimate, during periods when the footwear is not worn. In further embodiments, the plantar-pressure-based blood pressure estimate can be used to calibrate or recalibrate a PPG-based blood pressure estimation model running on the external wearable device.

In some embodiments, the one or more processors can receive one or more reference blood pressure measurements from an external instrument and calibrate the blood pressure estimation based on the reference measurements. In further embodiments, the one or more processors can periodically prompt the wearer to perform a reference blood pressure measurement for recalibration. In some embodiments, the footwear is selected from the group consisting of: a slipper, a shoe, an insole, a padded sock, and a sandal.

In some embodiments, the apparatus includes a wireless communication module that can transmit blood pressure estimates or plantar pressure data to a mobile computing device. In further embodiments, the apparatus includes a mobile application that can display blood pressure trends, generate alerts, and forward data to a cloud-based computing resource. In further embodiments, the one or more processors can estimate body weight from the weight-bearing component of the plantar pressure signal. In further embodiments, the apparatus includes an inertial measurement unit disposed within the footwear, and the one or more processors use inertial measurement data to distinguish weight-bearing pressure variations caused by gait from cardiovascular pulsatile variations. In embodiments, the apparatus includes one or more pairs of electrical impedance sensing electrodes disposed within the footwear that can inject an alternating current into tissue of the wearer's foot and measure a resulting voltage to determine a bioelectrical impedance at one or more excitation frequencies. In further embodiments, the one or more processors can estimate at least one body water parameter selected from: total body water, extracellular water, intracellular water, and a ratio of extracellular water to intracellular water, from the bioelectrical impedance measured at a plurality of excitation frequencies. In yet further embodiments, the one or more processors can detect at least one of: dehydration, edema, fluid retention, or fluid overload, based on deviations of the at least one body water parameter from a baseline value or from a configurable threshold.

In further embodiments, the one or more processors can estimate at least one body composition parameter selected from: fat mass, fat-free mass, lean mass, and skeletal muscle mass, from the bioelectrical impedance measured at a plurality of excitation frequencies. In further embodiments, the one or more processors can extract an impedance plethysmography waveform from time-varying impedance measurements at a sampling rate sufficient to resolve cardiac-synchronous pulsatile blood volume changes in plantar vasculature of the foot. In yet further embodiments, the one or more processors can calculate a blood pressure estimate based at least in part on both the impedance plethysmography waveform and the cardiovascular pulsatile component of the plantar pressure signal. In further embodiments, the electrical impedance sensing electrodes are arranged in a tetrapolar configuration including separate current-injection electrodes and voltage-sensing electrodes. In further embodiments, the electrical impedance sensing electrodes include at least one of: conductive textile electrodes on an insole surface, metallic or conductive polymer electrodes laminated into an insole structure, electrodes disposed on an inner surface of a footwear upper, or electrodes in contact with a conductive viscous liquid within a sealed enclosure.

In some embodiments, the one or more processors are can execute a predictive analytics engine that applies a machine learning model to longitudinal time-series data accumulated from the plantar pressure signal over a period of days to months to forecast at least one of: a future blood pressure trajectory over a prediction horizon, a probability of a hypertensive or hypotensive episode within the prediction horizon, or an abnormal nocturnal blood pressure dipping pattern. In further embodiments, the predictive analytics engine can compute a composite cardiovascular risk score by integrating a plurality of physiological parameters comprising at least two of: blood pressure variability metrics, heart rate variability indices, gait parameters, body composition trends, hydration status trends, pulse wave morphology features, and activity level metrics. In further embodiments, the predictive analytics engine can detect early signs of physiological deterioration by jointly analyzing multi-parameter trends, the physiological deterioration comprising at least one of: heart failure decompensation, autonomic dysfunction, renal function decline, medication nonadherence, or elevated fall risk. In further embodiments, the machine learning model of the predictive analytics engine comprises population-level model parameters learned from a cohort of wearers and individual-level model parameters adapted through transfer learning or fine-tuning based on the wearer's accumulated data. In further embodiments, the predictive analytics engine can generate personalized health insights by correlating physiological data of the wearer with contextual information including at least one of: time of day, activity state, medication schedule, or environmental conditions, and provides actionable recommendations or health summary reports.

In another aspect, the disclosed technology provides a method for noninvasive blood pressure estimation including: (a) measuring a time-varying plantar pressure signal from a foot of a wearer using one or more pressure sensors disposed within footwear, the plantar pressure signal including a weight-bearing component arising from the wearer's body weight and a cardiovascular pulsatile component arising from arterial blood pressure in plantar vasculature; (b) separating or identifying the cardiovascular pulsatile component from the plantar pressure signal; (c) determining an amplitude of the cardiovascular pulsatile component as a function of a magnitude of the weight-bearing component to characterize an oscillometric relationship; and (d) estimating at least one blood pressure parameter from the oscillometric relationship; and the method is performed without an inflatable cuff and without requiring deliberate user action beyond wearing the footwear.

In some embodiments, the method includes estimating the at least one blood pressure parameter, which includes identifying a weight-bearing pressure at which the pulsatile amplitude is maximized as an estimate of mean arterial pressure.

In some embodiments, estimating the at least one blood pressure parameter includes applying a machine learning model trained on simultaneously acquired plantar pressure data and reference blood pressure measurements.

In some embodiments, the method includes fusing the blood pressure estimate with a photoplethysmography-derived blood pressure estimate from an external wearable device to at least one of: (a) improve blood pressure estimation accuracy when both the footwear and the external wearable device are used simultaneously, or (b) provide continuous blood pressure monitoring during periods when the footwear is not worn.

In some embodiments, the method includes estimating heart rate and respiration rate from the plantar pressure signal.

In some embodiments, the one or more pressure sensors are disposed within a sealed, flexible enclosure in the footwear that is filled with a viscous liquid, and measuring the plantar pressure signal includes measuring hydrostatic pressure within the viscous liquid.

In some embodiments, the method includes: (a) injecting an alternating current into tissue of the wearer's foot via one or more pairs of electrical impedance sensing electrodes disposed within the footwear; (b) measuring a resulting voltage to determine a bioelectrical impedance at one or more excitation frequencies; and (c) estimating at least one of: a body water parameter, a body composition parameter, or an impedance plethysmography waveform, from the bioelectrical impedance.

In another aspect, the disclosed technology provides for a continuous blood pressure monitoring system including: (a) a pair of footwear articles, each including one or more flexible pressure sensors that can measure plantar pressure and an electronics module including signal processing circuitry, a wireless communication module, and a battery; (b) a mobile computing device configured to receive plantar pressure data from the pair of footwear articles via wireless communication; (c) software executing on the mobile computing device or on a cloud-based computing resource, the software configured to: (i) extract cardiovascular pulsatile components from the plantar pressure data, (ii) estimate blood pressure parameters from the extracted pulsatile components and corresponding weight-bearing pressures using an oscillometric analysis or a machine learning model, and (iii) present blood pressure trends and alerts to the wearer or a caregiver.

In some embodiments, the system includes a wrist-worn device providing photoplethysmography (PPG) data, wherein the software calculates the blood pressure estimate based at least in part on both the PPG data and the plantar pressure data and calibrates a PPG-based blood pressure model using the footwear-derived blood pressure estimates to extend monitoring to periods when the footwear is not worn. In some embodiments, the system includes one or more pairs of electrical impedance sensing electrodes disposed within at least one of the footwear articles, and capable of measuring bioelectrical impedance of the wearer's foot at one or more excitation frequencies, wherein the software estimates at least one of: body water parameters, body composition parameters, or impedance plethysmography waveforms from the bioelectrical impedance measurements. In some embodiments, comprising a predictive analytics engine executing on the cloud-based computing resource or the mobile computing device, the predictive analytics engine configured to analyze longitudinal time-series data accumulated from the footwear sensors to generate at least one of: blood pressure trajectory forecasts, cardiovascular risk scores, physiological deterioration alerts, or personalized health recommendations.

In certain embodiments, the insole or a portion thereof comprises a sealed, flexible enclosure filled with a viscous liquid, such as glycerine, silicone oil, or a biocompatible gel. One or more pressure sensors are mounted within or on the enclosure so as to be in hydraulic communication with the viscous liquid. The viscous liquid serves multiple functions: it provides cushioning and comfort comparable to a conventional gel insole; it transmits pressure from the plantar surface of the foot to the pressure sensor with reduced spatial localization, thereby acting as a mechanical low-pass filter that attenuates high-frequency gait impact artifacts while preserving the lower-frequency cardiovascular pulsatile signal; and it reduces the acoustic impedance mismatch between the foot and the sensor surface, improving coupling of arterial blood pressure pulsations into the measured pressure signal. Because the viscous liquid equilibrates pressure across the enclosed volume, a single pressure sensor within the enclosure can sense the aggregate plantar pressure over the contact area, simplifying the sensor architecture while maintaining sensitivity to blood-pressure-induced oscillations.

In certain embodiments, flexible weight-bearing pressure sensors are capacitive force transducers comprising flexible electrodes separated by a compressible dielectric, as described in co-pending patent applications by the same inventor. In other embodiments, the pressure sensors may comprise piezoresistive, piezoelectric, or other force or pressure sensing elements.

In certain embodiments, the signal processing separates the weight-bearing pressure component from the cardiovascular pulsatile component using filtering, demodulation, or adaptive separation techniques. The weight-bearing component provides the slowly varying applied pressure analogous to the cuff pressure in a conventional oscillometric instrument, while the pulsatile component provides the oscillometric signal from which blood pressure is estimated.

In certain embodiments, machine learning methods are employed to estimate blood pressure parameters from the plantar pressure signal. Training data may include simultaneous reference blood pressure measurements from a validated arm cuff, invasive catheter, or other reference instrument. The machine learning model may be trained on population-wide data and optionally fine-tuned or calibrated for an individual wearer.

In certain embodiments, photoplethysmography (PPG) signals acquired from a wrist-worn or other wearable device, or from the plantar sensor system itself, are fused with the plantar pressure data. Fusion of PPG data with the plantar pressure data may improve blood pressure estimation accuracy when the footwear is worn by providing complementary physiological information. Additionally, the footwear-based system provides high-accuracy blood pressure reference points that are used to calibrate or improve the accuracy of PPG-derived blood pressure estimates, thereby extending accurate blood pressure monitoring to periods when the footwear is not being worn and PPG is acquired from another device.

In certain embodiments, heart rate and respiration rate are simultaneously estimated from the plantar pressure signal, providing additional vital sign monitoring without additional sensors.

In certain embodiments, the footwear further comprises one or more electrical impedance sensing electrodes configured to inject a small alternating current into the foot and measure the resulting voltage to determine a bioelectrical impedance of tissue of the foot. The bioelectrical impedance measurements, taken at one or more frequencies, enable estimation of body water parameters including total body water (TBW), extracellular water (ECW), intracellular water (ICW), and the ratio ECW/ICW. Changes in the ECW/ICW ratio serve as indicators of dehydration, edema, fluid retention, or fluid shifts. Multi-frequency bioimpedance analysis further enables estimation of body composition parameters, including fat mass, lean mass (fat-free mass), and skeletal muscle mass, by exploiting the frequency-dependent behavior of current flow through intracellular and extracellular compartments. Additionally, time-varying impedance measurements at the cardiac frequency provide impedance plethysmography (IPG) waveforms that reflect pulsatile blood volume changes in the plantar vasculature, offering an independent measure of pulse timing and amplitude that may be fused with the pressure-based blood pressure estimates to improve accuracy. The impedance sensing electrodes may be integrated into the insole surface, the viscous-liquid-filled enclosure, or the footwear upper, and may share signal conditioning electronics with the pressure sensors.

In certain embodiments, the system further comprises an AI-based predictive analytics engine that analyzes longitudinal time-series data collected from the footwear sensors and optional external devices to generate predictive health insights. The predictive analytics engine applies machine learning models trained on population-level longitudinal health data to the wearer's accumulated history of blood pressure measurements, heart rate, heart rate variability, gait parameters, body weight, body composition, hydration status, and activity patterns to forecast future health events and trends. Predictive outputs include: short-term blood pressure trajectory forecasting to anticipate hypertensive or hypotensive episodes before they occur; cardiovascular risk scoring that integrates multiple physiological parameters into a composite risk index; detection of early-stage physiological deterioration patterns indicative of conditions such as heart failure decompensation, autonomic dysfunction, or renal impairment; and personalized health recommendations based on identified trends. The predictive analytics engine may execute on a cloud-based computing resource with access to population-level data for continual model refinement, or on a local device with a pre-trained model.

### BRIEF DESCRIPTION OF THE DRAWINGS

The drawings illustrate non-limiting example embodiments of the disclosed technology.

FIG. 1 is a block diagram illustrating the blood pressure estimation system comprising Footwear Sensor Assembly (12100), Signal Acquisition and Processing Electronics (12200), Blood Pressure Estimation Processing (12300), and optional External Wearable Devices (12400).

FIG. 2 illustrates the anatomy of the lateral plantar artery on the sole of the foot and the positioning of pressure sensors in the footwear relative to the artery.

FIG. 3 illustrates pulsatile modulation in the measured plantar arterial blood pressure signal because of the applied weight-bearing pressure.

FIG. 4 illustrates an oscillometric envelope constructed from the extracted pulsatile amplitudes as a function of the applied weight-bearing pressure, showing the identification of mean arterial pressure (MAP), systolic blood pressure (SBP), and diastolic blood pressure (DBP).

FIG. 5 is a block diagram of the signal processing pipeline for blood pressure estimation, including weight-bearing and pulsatile component separation, oscillometric envelope construction, and systolic, diastolic, and mean blood pressure parameter estimation.

FIG. 6 illustrates a machine learning based blood pressure estimator trained using reference blood pressure measurements.

FIG. 7 illustrates the fusion of footwear-based blood pressure estimates with PPG-derived estimates from a wrist-worn or other wearable device.

FIG. 8 illustrates a cross-section of the viscous-liquid-filled insole embodiment, showing the sealed flexible enclosure, the viscous liquid, the pressure sensor, and the sealed electrical feedthrough.

FIG. 9 illustrates the bioelectrical impedance sensing subsystem, including electrode placement on the insole for tetrapolar impedance measurement, the multi-frequency excitation and measurement circuit, and example outputs including body water compartment estimation, body composition analysis, and impedance plethysmography waveform.

FIG. 10 illustrates the AI-based predictive health analytics engine, showing the ingestion of longitudinal multi-modal sensor data, the predictive model architecture, and the generation of health forecasts, risk scores, and personalized alerts.

FIG. 11 illustrates a smart footwear embodiment with seamlessly integrated flexible force sensors connected to a battery-powered microcontroller communicating via Bluetooth Low Energy.

FIG. 12 illustrates the monitoring platform, including smart footwear transmitting sensor data to a mobile application that supports blood pressure trends, alerts, and parameter review, and forwards data to a cloud-based signals database and AI agents for population-level machine learning model training, event monitoring and predictive analytics.

FIG. 13 shows a force sensing computing system.

### DETAILED DESCRIPTION OF SOME EMBODIMENTS

The following description sets forth various specific details in order to provide a thorough understanding of the claimed subject matter. However, one skilled in the art will understand that the claimed subject matter may be practiced without these specific details.

The key insight underlying the disclosed technology is that the wearer's own body weight, transmitted through the sole of the foot, naturally compresses the lateral plantar artery and other plantar vasculature against the pressure-sensing surface in the footwear. This compression is functionally analogous to the externally applied pressure in a conventional oscillometric arm cuff instrument. As the wearer stands, shifts weight, walks, or performs other activities, the effective compression pressure on the plantar arteries varies over a range that has substantial overlap with the wearer's arterial blood pressure, thereby generating oscillometric information from which blood pressure parameters can be extracted.

### System Overview

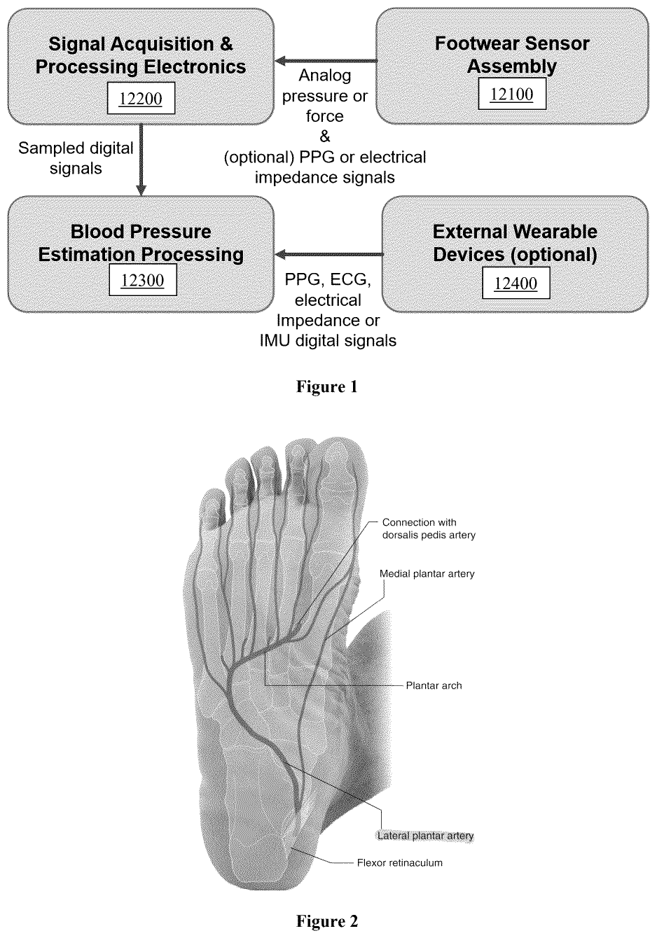

FIG. 1 is a block diagram for the blood pressure estimation system. The system comprises Footwear Sensor Assembly (12100), Signal Acquisition and Processing Electronics (12200), Blood Pressure Estimation Processor (12300), and optional External Wearable Device (12400).

Footwear Sensor Assembly (12100) includes one or more flexible pressure sensors integrated into the insole or sole of footwear such as slippers, shoes, padded socks, or removable insoles. The sensors are positioned to measure plantar pressure across the sole of the foot, including regions overlying or adjacent to the lateral plantar artery and its branches. In a preferred embodiment described further below, the insole comprises a sealed, flexible enclosure filled with a viscous liquid, such as glycerine, with one or more pressure sensors mounted within or on the enclosure in hydraulic communication with the viscous liquid. In other embodiments, the pressure sensors are flexible capacitive force transducers as described in co-pending patent applications by the same inventor, comprising flexible electrodes separated by a compressible dielectric layer. More generally, any pressure or force sensor technology capable of the required sensitivity and bandwidth may be used, including piezoresistive sensors, piezoelectric sensors, or optical pressure sensors.

Signal Acquisition and Processing Electronics (12200) includes analog signal conditioning, analog-to-digital conversion, and initial digital signal processing. This circuitry may be implemented on a small, low-power microcontroller or system-on-chip located within the footwear, connected to the sensors via flexible conductive traces or wires.

Blood Pressure Estimation Processor (12300) performs the computational algorithms for extracting blood pressure estimates from the digitized plantar pressure signals. This processor may be the same microcontroller as in Signal Acquisition and Processing Electronics (12200), or it may be a separate processor, a connected mobile phone, or a cloud-based computing resource.

Optional External Wearable Device (12400), such as a smart watch, may provide supplementary physiological signals including photoplethysmography (PPG), accelerometry, or electrodermal activity. Data from the External Wearable Device (12400) may be fused with the footwear sensor data to improve blood pressure estimation accuracy or extend estimation to periods when the footwear is not worn.

FIG. 12 illustrates a monitoring platform in which the smart footwear 23102 transmits sensor data to a mobile application 23104 that supports blood pressure trends, alerts, and parameter review, and forwards data to a cloud-based signals database and/or AI agents 23106 for population-level machine learning model training and continual model refinement.

### Oscillometric Principle Applied to Plantar Pressure

The disclosed technology exploits an oscillometric interaction between the wearer's body weight and the arterial blood pressure in the plantar vasculature. This interaction is fundamentally analogous to the interaction between cuff pressure and brachial arterial blood pressure in a conventional oscillometric arm cuff instrument.

When the wearer stands or walks, the body weight generates a distributed pressure field on the sole of the foot. A portion of this pressure acts on the lateral plantar artery and other plantar arteries, partially or fully compressing them against the underlying bony structures and the sensor surface. The degree of arterial compression depends on the local weight-bearing pressure p_w(t) relative to the internal arterial blood pressure p_b(t).

Under quasi-hydrostatic conditions, the total pressure measured by a sensor element positioned over or near a plantar artery is modeled as:

$$p ( t ) = {p}_{w} ( t ) + {P}_{max} exp ( - \frac{1}{2} {( \frac{{p}_{w} ( t ) - {P}_{map}}{{P}_{s} - {P}_{s}} )}^{2} ) {p}_{b} ( t ) \qquad ( Eq . 1 )$$

where: p_{w}(t) is the weight-bearing pressure component, varying with posture, weight distribution, and gait; p_{b}(t) is the arterial blood pressure waveform, pulsating at the heart rate; Pₘₐₚ is the mean arterial pressure; Pₛ is the systolic blood pressure (maximum of p_{b}(t)); P_{d} is the diastolic blood pressure (minimum of p_{b}(t)); Pₘₐₓ is the peak amplitude of the oscillometric compliance curve; and the Gaussian function models the arterial wall compliance, which determines how strongly the blood pressure pulsations couple into the measured pressure signal as a function of the transmural pressure (p_{w}(t)−Pₘₐₚ).

The Gaussian compliance function reaches its maximum when the applied weight-bearing pressure p_{w}(t) equals the mean arterial pressure Pₘₐₚ. The pulsatile amplitude is small when p_{w}(t) is much greater than systolic pressure Pₛ (artery fully compressed) or much less than diastolic pressure P_{d} (artery fully open with minimal wall compliance variation). The pulse pressure (Pₛ−P_{d}) determines the width of the compliance curve.

In a conventional arm cuff instrument, the applied pressure p_{w}(t) is a smooth, controlled ramp generated by an air pump. In the disclosed technology, p_{w}(t) is the naturally varying weight-bearing pressure generated by the wearer's body weight during standing, walking, or other activities. Although p_{w}(t) is not a smooth controlled signal, it contains both slowly varying components (from postural shifts, weight transfer between feet) and impulsive or periodic components (from gait cycles) that together cause p_{w}(t) to traverse a range of pressures spanning the arterial blood pressure range. This provides the oscillometric information necessary to estimate blood pressure.

### Signal Processing for Blood Pressure Estimation

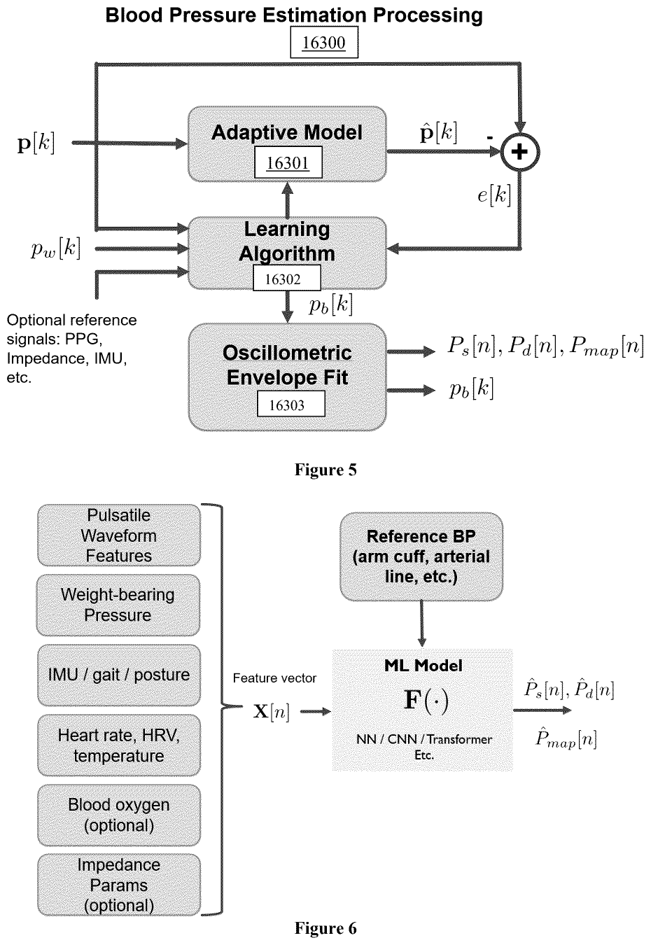

FIG. 5 illustrates the signal processing pipeline. The processing comprises the following stages:

### Pressure Signal Acquisition

The one or more pressure sensors in the footwear produce a time-varying electrical signal proportional to the local plantar pressure. This signal is conditioned by analog electronics (amplification, anti-aliasing filtering) and digitized by an analog-to-digital converter at a sampling rate sufficient to resolve the cardiac pulsatile waveform. A sampling rate of 40 Hz or higher is preferred, with 100 Hz or higher providing improved waveform fidelity.

### Weight-Bearing and Pulsatile Component Separation

The digitized total pressure signal p[k] contains both a weight-bearing component p_{w}[k] and a cardiovascular pulsatile component p_{b}[k] related to each other via Eq. 1. FIG. 5 shows Blood Pressure Estimation (12300) implemented using an adaptive process where parameters of Adaptive Model (16301) are estimated using Learning Algorithm (16302). The weight bearing ground reaction force (GRF) signal p_{w}[k] is a main reference signal. The adaptive model supports removal of gait-induced and weight-bearing pressure variations, leaving the cardiovascular component p_{b}[k] as the residual. Signal p_{w}[k] may be obtained from a flexible capacitive force transducer as described in co-pending patent applications by the same inventor. Because the GRF signal from the co-pending force sensor directly measures the total weight-bearing load on the insole, it provides a particularly effective reference for the adaptive filter, enabling more complete separation of p_{w}[k] and improved isolation of the small cardiovascular pulsatile component p_{b}[k]. Reference signals may also include photoplethysmography (PPG), accelerometer or inertial measurement unit (IMU) data, or impedance measurement data. Learning Algorithm (16302) may update adaptive coefficients or model parameters in real time using a least-mean-squares (LMS), recursive least-squares (RLS), or similar algorithm.

Linear low-pass or high-pass filters can also support signal separation. The weight-bearing component p_{w}[k] varies at frequencies associated with postural changes and gait (typically below 5 Hz), while the cardiac pulsatile component p_{b}[k] has energy centered at the heart rate frequency (typically 0.8-3 Hz) and its harmonics.

Data-driven decomposition methods such as empirical mode decomposition or wavelet decomposition can also support separation of the signal components using intrinsic mode functions or wavelet coefficients, from which the cardiac component could be identified and isolated based on its characteristic frequency and morphology.

### Oscillometric Envelope

In addition to potential direct measurement as adaptive model parameters, the blood pressure parameters Pₘₐₓ, Pₘₐₚ, Pₛ, and P_{D} can be found by fitting an envelope to a histogram of pulsatile component sample p_{b}[k] peaks versus weight p_{w}[k]. Once the pulsatile component p_{b}[k] is extracted, its amplitude Aₖ (peak-to-peak or root-mean-square) is computed for each cardiac cycle or over a short sliding window. This amplitude is paired with the corresponding value of the weight-bearing pressure p_{w}[k] to construct a histogram of data points:

$${\{ ( {p}_{w} [ k ] , {A}_{k} ) \}}_{k = 1}^{K} \qquad ( Eq . 2 )$$

These data points are accumulated over a time window (e.g., 10 seconds to several minutes) during which the weight-bearing pressure traverses a sufficient range.

A parametric or nonparametric curve can be fit to these data points to form the oscillometric envelope (for example, in the oscillometric envelope fit 16303). A Gaussian model as in Eq. 1 may be used,

$$A ( {p}_{w} ) = {P}_{max} exp ( - \frac{1}{2} {( \frac{{p}_{w} - {P}_{map}}{{P}_{s} - {P}_{s}} )}^{2} ) \qquad ( Eq . 3 )$$

or a spline or kernel regression may be used.

### Blood Pressure Parameter Estimation

From the oscillometric envelope, blood pressure parameters are estimated as follows:
(a) Mean Arterial Pressure (MAP): The weight-bearing pressure at which the oscillometric envelope reaches its maximum amplitude:

$$\text{?} = arg {max}_{{p}_{w}} \hat{A} ( {p}_{w} ) \qquad ( Eq . 4 )$$

where Â(p_{w}) is the fitted envelope function.
(b) Systolic Blood Pressure (SBP): Estimated from the weight-bearing pressure on the high-pressure side of the envelope where the amplitude falls to a characteristic fraction rₛ(typically 0.5-0.7) of the peak amplitude:

$$\text{?} : \hat{A} ( \text{?} ) = {r}_{s} \cdot \hat{A} ( \text{?} ) , \qquad ( Eq . 5 )$$

$$\text{?} > \text{?}$$

(c) Diastolic Blood Pressure (DBP): Estimated from the weight-bearing pressure on the low-pressure side of the envelope where the amplitude falls to a characteristic fraction r_{d} (typically 0.6-0.8) of the peak amplitude:

$$\text{?} : \hat{A} ( \text{?} ) = {r}_{s} \cdot \hat{A} ( \text{?} ) , \qquad ( Eq . 6 )$$

$$\text{?} < \text{?}$$

The characteristic ratios rₛ and r_{d} may be fixed empirically determined constants, or they may be parameters of a machine learning model trained on reference data.

### Machine Learning Based Blood Pressure Estimation

FIG. 6 illustrates an alternative or supplementary embodiment in which a machine learning model estimates blood pressure parameters directly from features of the plantar pressure signal without explicitly constructing an oscillometric envelope.

Let x[n] denote a feature vector extracted from the plantar pressure signals at time n. The feature vector may include: time-domain features of the pulsatile waveform (amplitude, width, rise time, systolic-diastolic ratio); frequency-domain features (spectral power at heart rate and harmonics); the current weight-bearing pressure level and its recent history; inertial measurement unit (IMU) data indicating posture and activity state; heart rate estimated from the pulsatile component; ambient temperature and other environmental measurements; bioelectrical impedance parameters (resistance, reactance, Cole model parameters) at one or more frequencies, when impedance sensing electrodes are present; and impedance plethysmography waveform features (pulse amplitude, timing, morphology), when impedance sensing electrodes are present.

A machine learning model F maps the feature vector to blood pressure estimates:

$$[ {\hat{P}}_{s} [ n ] , {\hat{P}}_{d} [ n ] , {\hat{P}}_{map} [ n ] ] = F ( x [ n ] , x \{ n - 1 ] , … ) \qquad ( Eq . 7 )$$

The model F may be a neural network (e.g., recurrent neural network, transformer, or convolutional neural network), a gradient-boosted decision tree, a Gaussian process, or any other suitable regression model. The model takes as input the current and past feature vectors to account for temporal dynamics.

Training is performed using simultaneously collected plantar pressure data and reference blood pressure measurements from a validated arm cuff or arterial line. The training dataset may include data from many individuals (population-level training) and may be fine-tuned for a specific individual using a small number of calibration measurements.

In a preferred embodiment, the system periodically prompts the wearer to perform a reference arm cuff measurement for recalibration. The recalibration data is used to update or fine-tune the machine learning model, compensating for physiological changes, sensor aging, or other drift.

Fusion with Photoplethysmography

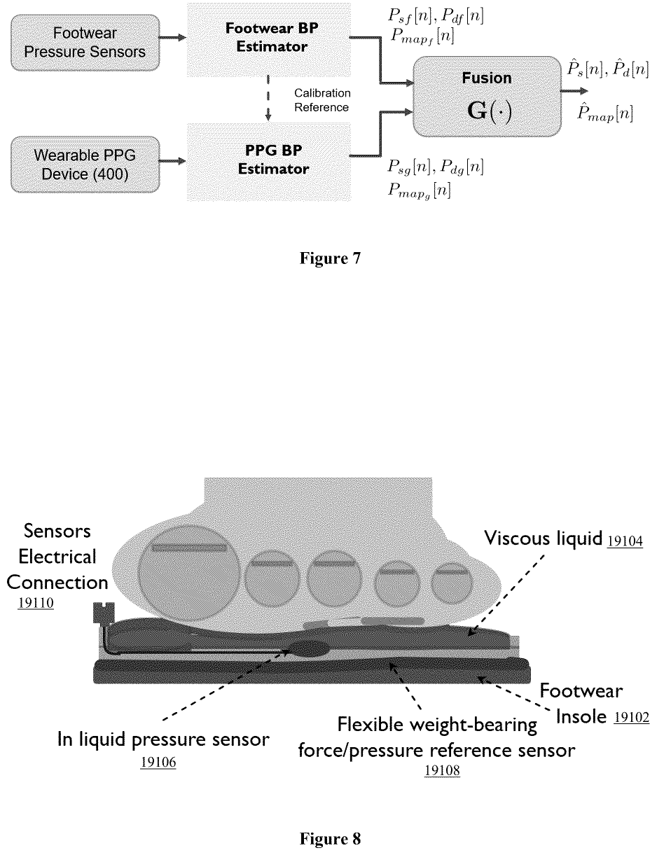

FIG. 7 illustrates the fusion of footwear-based blood pressure estimates with photoplethysmography (PPG) data from a wrist-worn or other wearable device.

PPG signals acquired from a smart watch or fitness tracker provide pulse wave information that correlates with blood pressure through pulse transit time, pulse wave velocity, or pulse wave morphology features. However, the correlation between PPG features and true arterial blood pressure is subject to drift and confounding factors, requiring frequent recalibration against a reference standard to maintain accuracy.

The fusion of PPG data with plantar pressure data serves two purposes. First, when both the footwear and the wrist-worn device are worn simultaneously, the complementary physiological information from the two modalities may improve blood pressure estimation accuracy beyond what either modality achieves alone. For example, the PPG waveform provides pulse transit time and morphology features that encode information about arterial stiffness and peripheral vascular resistance, while the plantar pressure provides direct oscillometric information; combining these inputs in a joint estimation model can reduce uncertainty. Second, the disclosed technology provides a uniquely convenient and frequent source of reference blood pressure measurements. Each time the wearer puts on the footwear and stands or walks for a sufficient duration, the footwear system generates one or more blood pressure estimates. These estimates serve as calibration points for the PPG-based model running on the wrist-worn device, enabling the PPG model to maintain accuracy between footwear wearing periods, including during sleep or sedentary activities when the footwear is not worn.

The fusion model is:

$${\hat{P}}_{fused} [ n ] = G ( {\hat{P}}_{foot} [ n ] , {\hat{P}}_{ppg} [ n ] ) \qquad ( Eq . 8 )$$

where \hat{P}_{foot}[n] is the footwear-based estimate (available when footwear is worn), \hat{P}ₚₚg [n] is the PPG-based estimate (available when the wrist device is worn). The fusion function G weights the two estimates based on their expected accuracy, with the footwear estimate receiving higher weight when available and the PPG estimate receiving higher weight when the footwear is not worn but was recently calibrated.

### Additional Vital Signs

The plantar pressure signal contains additional physiological information beyond blood pressure.

Heart Rate: The frequency of the pulsatile component directly provides heart rate. Beat-to-beat heart rate variability (HRV) can also be extracted from the inter-beat intervals of the pulsatile waveform.

Respiration Rate: Respiration modulates both the amplitude and frequency of the cardiac pulsatile waveform (respiratory sinus arrhythmia) and may also cause low-frequency modulation of the weight-bearing pressure. These modulations can be extracted to estimate respiration rate.

### Bioelectrical Impedance Sensing

In certain embodiments, the footwear includes one or more pairs of electrical impedance sensing electrodes disposed on or within the insole, the insole surface, the footwear upper, or the viscous-liquid-filled enclosure. These electrodes are configured in a two-electrode or four-electrode (tetrapolar) arrangement, with current-injection electrodes and voltage-sensing electrodes positioned to establish a current path through plantar tissue of the foot.

### Impedance Measurement Principle

A small alternating current I(f) at one or more excitation frequencies f is injected through the current electrodes, and the resulting voltage V(f) across the sensing electrodes is measured. The complex bioelectrical impedance is:

$$Z ( f ) = \frac{V ( f )}{I ( f )} = R ( f ) + jX ( f ) \qquad ( Eq . 9 )$$

where R(f) is the resistance (real part) and X(f) is the reactance (imaginary part). The excitation current is small (typically 100-800 μA) and at frequencies between 1 kHz and 1 MHz, well within safe limits for continuous measurement.

At low frequencies (below approximately 50 kHz), the alternating current flows predominantly through the extracellular fluid because cell membranes act as capacitive barriers. At higher frequencies (above approximately 100 kHz), the current penetrates cell membranes and flows through both extracellular and intracellular compartments. This frequency-dependent behavior allows multi-frequency or bioimpedance spectroscopy (BIS) measurements to distinguish extracellular and intracellular fluid volumes.

### Body Water Estimation

From the multi-frequency impedance measurements, a Cole model or equivalent circuit model may be fitted:

$$Z ( f ) = {R}_{∞} + \frac{{R}_{0} - {R}_{∞}}{1 + {( j 2 π f τ )}^{α}} \qquad ( Eq . 10 )$$

where R₀ is the resistance at zero frequency (reflecting extracellular resistance only), R_{∞} is the resistance at infinite frequency (reflecting combined extracellular and intracellular resistance), tau is the characteristic time constant, and α is the dispersion parameter (typically 0.5-1.0).

From the fitted model parameters, the following body water compartments are estimated:
(a) Extracellular water (ECW): Inversely related to R₀. A decrease in R₀ indicates an increase in extracellular fluid volume, as may occur in edema, fluid retention, or congestive heart failure.
(b) Intracellular water (ICW): Derived from the difference between intracellular resistance Rᵢ(computed from R₀ and R_{∞}) and R₀.
(c) Total body water (TBW): The sum of ECW and ICW.
(d) ECW/ICW ratio: The ratio of extracellular to intracellular water. An elevated ECW/ICW ratio may indicate edema, fluid retention, or fluid overload. A depressed ratio may indicate dehydration or intracellular overhydration.

### Dehydration, Edema, and Fluid Retention Detection

The one or more processors are configured to track the ECW/ICW ratio and related impedance parameters over time and to detect deviations from the wearer's baseline values. Specific clinical indicators include:
(a) Dehydration: Characterized by increased resistance at all frequencies (indicating decreased total body water), with a relative decrease in the ECW/ICW ratio as extracellular fluid is preferentially lost.
(b) Edema and fluid retention: Characterized by decreased extracellular resistance R_0 (indicating increased ECW), with an elevated ECW/ICW ratio. Pedal edema is particularly well-suited for detection via foot impedance measurements, as gravitational fluid accumulation occurs preferentially in the lower extremities.
(c) Congestive heart failure (CHF) decompensation: Progressive fluid overload in CHF patients manifests as increasing ECW and increasing ECW/ICW ratio, detectable days before clinical symptoms such as dyspnea or weight gain become apparent. Early detection enables timely medical intervention.

The system may generate alerts when impedance-derived fluid parameters exceed configurable thresholds or exhibit trends indicative of clinical deterioration.

### Body Composition Estimation

Multi-frequency bioimpedance measurements additionally enable estimation of body composition:
(a) Fat-free mass (lean mass): Because lean tissue (muscle, organs) is highly hydrated and electrically conductive, while adipose tissue has low water content and high impedance, the measured bioimpedance is primarily determined by the volume and distribution of lean tissue. Using established empirical relationships between impedance, height, weight, and fat-free mass, the system estimates lean body mass.
(b) Fat mass: Estimated as the difference between total body weight (measured by the pressure sensors or by an external scale) and fat-free mass.
(c) Skeletal muscle mass: Estimated using segmental or whole-body impedance in conjunction with demographic data and machine learning models.

Body composition tracking over time enables monitoring of sarcopenia (muscle loss) in elderly wearers, nutritional status, and the effects of exercise or dietary interventions.

### Impedance Plethysmography

The bioimpedance measurement, when acquired at a sufficiently high sampling rate (e.g., 50 Hz or higher at a single excitation frequency), captures time-varying impedance changes caused by pulsatile blood volume changes in the plantar vasculature. This impedance plethysmography (IPG) waveform delta_Z(t) reflects the cardiac-synchronous expansion and contraction of blood vessels in the foot:

$$Δ Z ( t ) = {Z}_{0} - Z ( t ) \approx \frac{{ρ L}^{2}}{{V}_{0}^{2}} \cdot {Δ V}_{blood} ( t ) \qquad ( Eq . ll )$$

where Z₀ is the baseline impedance, ρ is the blood resistivity, L is the distance between sensing electrodes, V₀ is the baseline tissue volume, and ΔV_{blood}(t) is the pulsatile blood volume change.

The IPG waveform provides: (a) An independent measure of cardiac pulse timing, which may be used to validate or improve beat detection from the pressure signal; (b) Pulse amplitude information proportional to the local blood volume change, offering an independent indicator of peripheral vascular perfusion; (c) Pulse transit time from the heart to the foot when combined with an electrocardiogram (ECG) or PPG signal from a wrist-worn device, which correlates with arterial stiffness and blood pressure; (d) A signal that may be fused with the pressure-based oscillometric data to improve blood pressure estimation accuracy, particularly in conditions where the pressure signal quality is degraded (e.g., low weight-bearing pressure during sitting).

### Electrode Integration

The impedance sensing electrodes may be implemented as: (a) Conductive textile or fabric electrodes woven or printed onto the insole surface or sock liner, providing dry-contact coupling with the plantar skin; (b) Metallic or conductive polymer electrodes laminated into the insole structure; (c) Electrodes disposed on the inner surface of the footwear upper, contacting the dorsal surface of the foot; (d) In the viscous-liquid-filled insole embodiment, electrodes in contact with the conductive viscous liquid (e.g., glycerine with ionic additives), using the liquid as a coupling medium to improve electrode-skin contact impedance.

The impedance measurement electronics may be integrated into the same electronics module (12200) as the pressure sensor signal conditioning, sharing the microcontroller, wireless communication module, and battery. The impedance measurement may be performed intermittently (e.g., once per minute for body water assessment) or continuously (for impedance plethysmography), with the measurement cadence configurable by the system or user.

### Sensor Placement and Footwear Integration

In a preferred embodiment, the pressure is measured inside a viscous-liquid-filled insole enclosure as described below. By Pascal's principle, pressure applied anywhere on the surface of the enclosed liquid is transmitted equally and undiminished throughout the liquid. Consequently, a single pressure sensor mounted at any location within or on the enclosure, for example on the bottom interior wall, senses the aggregate plantar pressure over the entire foot contact area without requiring spatial distribution of multiple sensors. This substantially simplifies the sensor architecture, wiring, and electronics while preserving full sensitivity to both the weight-bearing component and the cardiovascular pulsatile component needed for blood pressure estimation.

In alternative embodiments using solid-dielectric or dry sensor architectures, the pressure or force sensors may be distributed across the insole to cover multiple regions of the foot. At least one sensor or sensor region is positioned to overlie or be adjacent to the lateral plantar artery, which runs along the lateral aspect of the sole of the foot. Additional sensors may cover the heel, metatarsal heads, and medial arch regions.

The sensors are integrated into the footwear in a manner that maintains comfort and does not significantly alter the feel or appearance of the footwear. In solid-dielectric embodiments, the sensors are thin, flexible capacitive transducers laminated into the insole structure. Conductive traces connect the sensors to a small electronics module located unobtrusively in the footwear, such as the heel or arch region or enclosed in uppers above the foot.

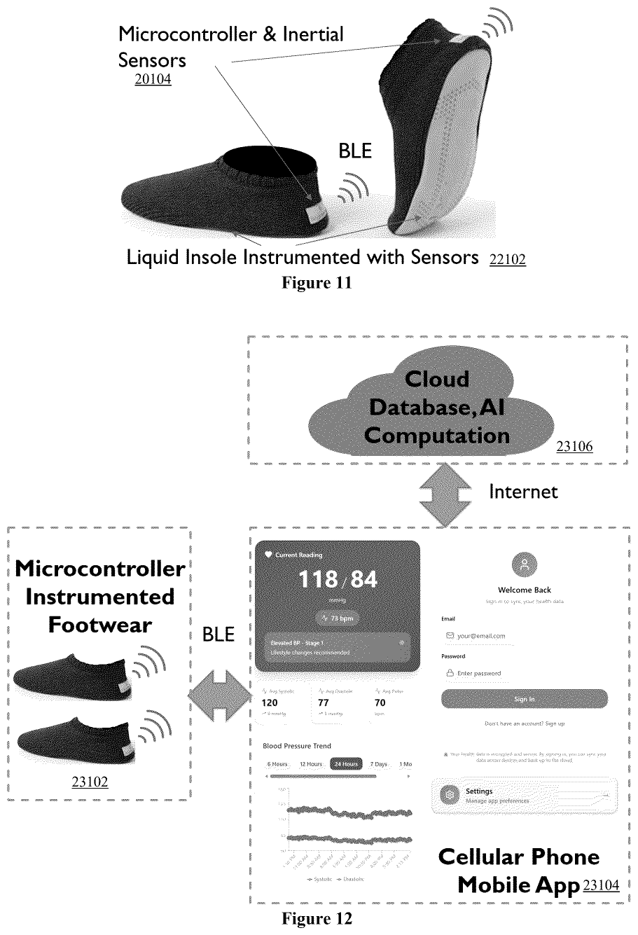

The electronics module includes a microcontroller, analog front-end circuitry, a wireless communication module (e.g., Bluetooth Low Energy), and a battery. The microcontroller performs initial signal processing and transmits data wirelessly to a mobile phone application for further processing, display, and storage. FIG. 11 illustrates a smart slipper embodiment in which flexible force sensors 22102 are seamlessly integrated into the slipper and connected to a small battery-powered microcontroller 22104 communicating via Bluetooth Low Energy. The small battery-powered microcontroller 22104 can also include inertial sensors.

### Viscous-Liquid-Filled Insole Embodiment

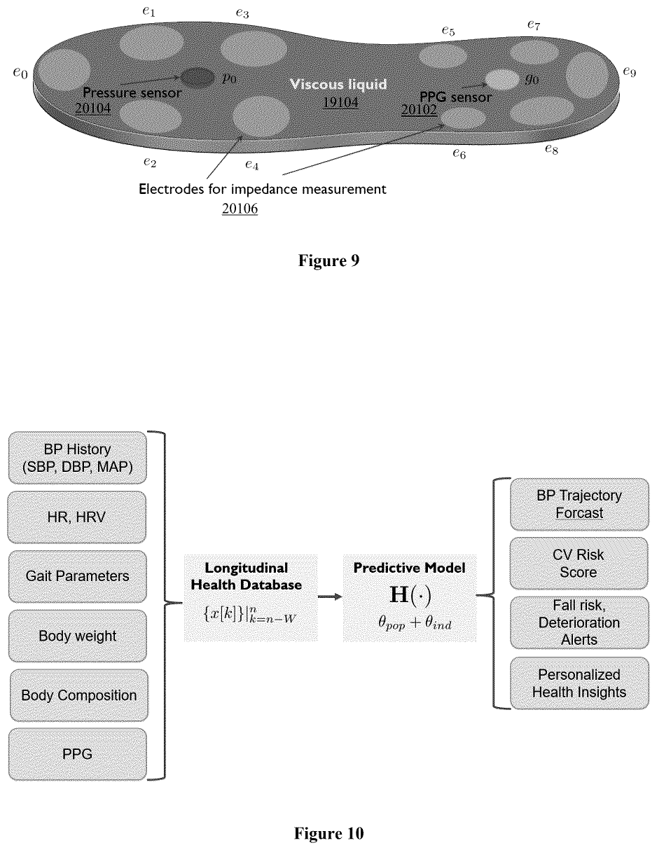

FIG. 8 illustrates an embodiment in which the insole 19102 or a portion thereof comprises a sealed, flexible enclosure filled with a viscous liquid 19104. The insole 19102 can also include an in-liquid pressure sensor 19106 and a flexible, weight-bearing force/pressure reference sensor 19108, and electrical connections 19110 in communication with both sensors. Suitable viscous liquids include glycerine, silicone oil, mineral oil, or a biocompatible gel. The enclosure is formed from a flexible, liquid-impermeable membrane material such as thermoplastic polyurethane (TPU), silicone elastomer, or a laminated polymer film. The enclosure is shaped and dimensioned to conform to the insole cavity of the footwear and is sealed at its edges to prevent leakage. FIG. 9 illustrates an isometric view of an insole, including a viscous liquid 19104, as well as PPG sensor 20102, pressure sensor 20104, and electrodes for impedance measurements 20106.

One or more pressure sensors are mounted within or on the enclosure so as to be in hydraulic communication with the viscous liquid. In one sub-embodiment, a single pressure sensor is mounted on the interior wall of the enclosure, for example on the bottom surface, and is fully immersed in the viscous liquid. In another sub-embodiment, the pressure sensor is mounted on an exterior wall of the enclosure such that a flexible diaphragm portion of the enclosure wall transmits the internal liquid pressure to the sensor. In either case, the sensor measures the hydrostatic pressure within the viscous liquid, which reflects the aggregate plantar pressure applied by the foot over the contact area.

The viscous-liquid-filled insole offers several advantages for blood pressure estimation:
(a) Mechanical impedance matching: The viscous liquid reduces the acoustic and mechanical impedance mismatch between the soft tissue of the foot and the pressure sensor. This improved coupling enhances transmission of small arterial blood pressure pulsations from the plantar vasculature through the insole to the sensor, increasing the signal-to-noise ratio of the cardiovascular pulsatile component.
(b) Spatial pressure integration: Because the liquid equilibrates pressure hydrostatically across the enclosed volume, the sensor measures the area-averaged plantar pressure over the entire contact region rather than a localized point pressure. This provides inherent spatial integration, so that a single sensor suffices to capture the aggregate oscillometric interaction between the body weight and the plantar arteries across the full foot contact area. The spatial averaging also reduces sensitivity to exact foot placement and to localized pressure concentrations from bony prominences.
(c) Mechanical low-pass filtering: The viscosity of the liquid attenuates high-frequency mechanical transients, such as heel-strike impacts during walking, while preserving the lower-frequency cardiovascular pulsatile signal (typically 0.8-3 Hz fundamental). This intrinsic mechanical filtering simplifies subsequent electronic signal processing for pulsatile component extraction.
(d) Comfort: A viscous-liquid-filled or gel insole provides cushioning and a comfortable feel comparable to commercially available gel insoles, making it suitable for extended daily wear without discomfort.

In a preferred sub-embodiment, the pressure sensor within the viscous-liquid-filled enclosure is a piezoresistive or capacitive pressure transducer with sufficient sensitivity to resolve pressure variations on the order of 10-100 Pa, which is the expected magnitude of the blood-pressure-induced pulsatile component at the plantar surface. The sensor is connected to Signal Acquisition and Processing Electronics (12200) via a sealed feedthrough or flexible lead that exits the enclosure without compromising the liquid seal.

The viscous-liquid-filled insole embodiment may be combined with any of the signal processing, machine learning, PPG fusion, or calibration methods described in the preceding subsections. The viscous liquid may optionally contain a dye or be transparent to facilitate visual leak detection. The enclosure may include one or more internal baffles to control liquid flow distribution or to provide structural support preventing the insole from bottoming out under high loads.

### Calibration

Initial calibration of the blood pressure estimation may be performed at the factory using population-level machine learning models. The wearer may perform one or more reference blood pressure measurements using a validated arm cuff instrument in order to confirm or update factory calibration. The reference measurements are input to the mobile phone application and used to calibrate or fine-tune the estimation model for the individual wearer.

Periodic recalibration may be prompted by the application at configurable intervals (e.g., weekly or monthly) or when the system detects that estimation confidence has decreased.

### AI-Based Predictive Health Analytics

FIG. 10 illustrates an AI-based predictive health analytics engine that operates on the longitudinal time-series data collected by the footwear sensor system and optional external devices.

### Longitudinal Data Accumulation

The system continuously accumulates a wearer-specific health database comprising time-stamped measurements of: blood pressure (SBP, DBP, MAP) estimated from the plantar pressure signal; heart rate and heart rate variability derived from the pulsatile component; body weight estimated from the weight-bearing component; gait parameters (cadence, stride variability, stance duration, weight-transfer asymmetry) derived from the plantar pressure and inertial measurement signals; body composition parameters (fat mass, lean mass, skeletal muscle mass) and hydration parameters (ECW, ICW, TBW, ECW/ICW ratio) from bioelectrical impedance measurements when available; activity level and sleep pattern metrics; ambient temperature; and any supplementary physiological data from external wearable devices such as PPG-derived oxygen saturation or electrodermal activity. This longitudinal database grows over days, weeks, months, and years of continuous wear, capturing diurnal patterns, responses to medications, seasonal variations, and long-term physiological trends.

### Predictive Model Architecture

A predictive model H maps the wearer's accumulated longitudinal data to one or more predictive health outputs:

$${y}_{pred} [ n ] = H ( {\{ x [ k ] \}}_{k = n - W}^{n} , {θ}_{pop} , {θ}_{ind} ) \qquad ( Eq . 12 )$$

where Y_{pred}[n] is a vector of predictive outputs at time n, x[k] for k from n−W to n is the feature history over a lookback window W (ranging from hours to months depending on the prediction task), θₚₒₚ are population-level model parameters learned from a large cohort of wearers, and θ_{ind} are individual-level parameters adapted through transfer learning or fine-tuning on the specific wearer's data.

The predictive model H may comprise one or more of: a temporal convolutional network operating on multi-channel time-series inputs; a transformer-based sequence model with attention mechanisms that learn to weight clinically relevant temporal patterns; a recurrent neural network (LSTM or GRU) capturing long-range temporal dependencies; an ensemble of gradient-boosted decision trees operating on engineered temporal features; or a hybrid architecture combining neural feature extraction with statistical survival models for event prediction.

### Blood Pressure Trajectory Forecasting

The predictive analytics engine forecasts the wearer's blood pressure trajectory over a configurable prediction horizon (e.g., 1 hour, 6 hours, 24 hours, or 7 days ahead). By learning the wearer's characteristic blood pressure response patterns to activities, postures, meals, medications, and circadian rhythms, the model anticipates upcoming blood pressure excursions. Specifically, the system may predict:
(a) Hypertensive episodes: The probability that SBP will exceed a configurable threshold (e.g., 180 mmHg) within the prediction horizon, enabling the wearer or caregiver to take preemptive action such as administering antihypertensive medication or reducing activity.
(b) Hypotensive episodes: The probability that SBP will fall below a configurable threshold (e.g., 90 mmHg), which may be relevant for wearers on antihypertensive medications, elderly wearers prone to orthostatic hypotension, or wearers with autonomic dysfunction.
(c) Nocturnal blood pressure dipping: Prediction of abnormal nocturnal dipping patterns (non-dipping, reverse dipping, or extreme dipping), which are independent cardiovascular risk factors, based on daytime blood pressure patterns and activity data.

### Cardiovascular Risk Scoring

The predictive analytics engine computes a composite cardiovascular risk score by integrating multiple physiological parameters measured by the footwear system. The risk score incorporates: (a) Blood pressure variability metrics (visit-to-visit variability, morning surge magnitude, weighted standard deviation); (b) Resting heart rate trends and heart rate variability indices (SDNN, RMSSD, pNN50, low-frequency/high-frequency ratio); (c) Gait parameters that correlate with cardiovascular health (gait speed, stride variability, asymmetry); (d) Body composition trends (progressive loss of lean mass, increasing fat mass); (e) Hydration status trends (fluid retention patterns, ECW/ICW ratio trajectory); (f) Pulse wave features extracted from the pulsatile pressure waveform or IPG waveform (augmentation index, reflection index, stiffness index) that reflect arterial compliance; (g) Activity level and sedentary behavior metrics.

The composite risk score is updated continuously and presented to the wearer or caregiver as a summary health metric. Clinically significant changes in the risk score trigger alerts. The risk scoring model may be calibrated against established cardiovascular risk calculators (e.g., Framingham Risk Score, ASCVD Pooled Cohort Equations) using population-level outcome data, thereby providing an equivalent or improved risk estimate derived from continuously measured physiological data rather than from intermittent clinical measurements.

### Early Deterioration Detection

The predictive analytics engine monitors multi-parameter trends to detect early signs of physiological deterioration that may precede clinical events:
(a) Heart failure decompensation: By jointly analyzing trends in blood pressure, fluid status (ECW, ECW/ICW ratio), body weight, heart rate, and activity level, the system detects the gradual physiological signature of fluid overload and declining cardiac output that typically precedes hospitalization for heart failure by days to weeks.
(b) Autonomic dysfunction: Abnormal patterns in heart rate variability, blood pressure variability, and postural blood pressure responses (detected via gait-associated blood pressure changes) may indicate autonomic neuropathy or other neurological conditions.
(c) Renal function decline: Progressive changes in fluid distribution (increasing ECW/ICW ratio), blood pressure patterns (resistant hypertension), and body composition may indicate declining renal function and prompt further clinical evaluation.
(d) Medication nonadherence or inefficacy: Unexpected blood pressure elevations that deviate from the wearer's learned medication response pattern may indicate missed doses or loss of medication efficacy, enabling timely intervention.
(e) Fall risk assessment: Deteriorating gait parameters (decreased gait speed, increased stride variability, asymmetry), combined with orthostatic blood pressure changes and sarcopenia indicators from body composition tracking, provide an integrated fall risk assessment particularly relevant for elderly wearers.

### Personalized Health Insights and Recommendations

The predictive analytics engine generates personalized, actionable health insights by correlating the wearer's physiological data with contextual information. Examples include: (a) Identifying specific activities, times of day, dietary patterns, or environmental conditions associated with elevated blood pressure in the individual wearer; (b) Quantifying the blood pressure response to antihypertensive medications and providing medication timing optimization suggestions; (c) Detecting the physiological impact of lifestyle changes (exercise programs, dietary modifications, weight loss) on blood pressure and other vital signs, providing positive reinforcement or adjustment recommendations; (d) Generating periodic health summary reports for the wearer's healthcare provider, highlighting trends, anomalies, and risk changes.

The predictive analytics engine may execute on a cloud-based computing resource where population-level data is available for model training and continual refinement. Alternatively, a pre-trained and compressed model may execute locally on the wearer's mobile device or on an edge computing device, with periodic model updates downloaded from the cloud. Privacy-preserving techniques such as federated learning or differential privacy may be employed to train population-level models without centralizing individual health data.

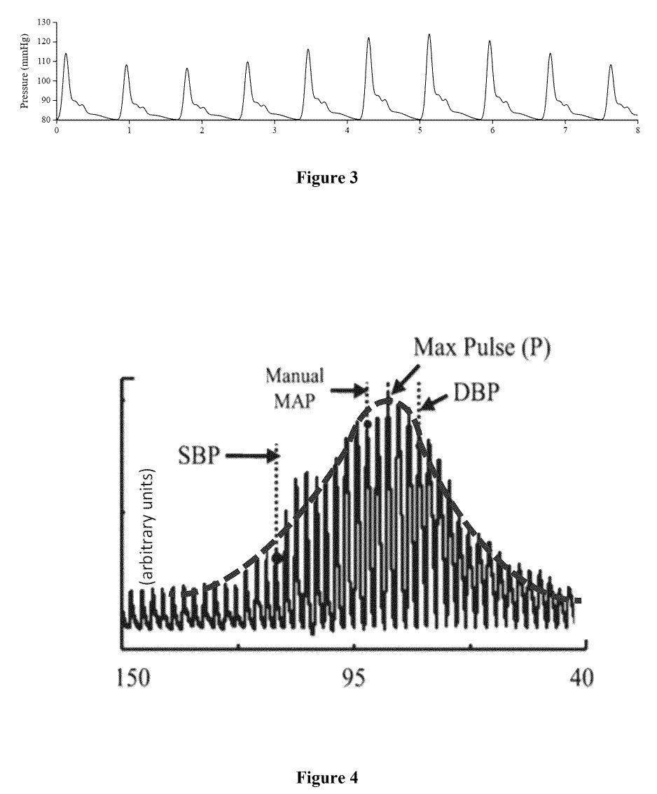

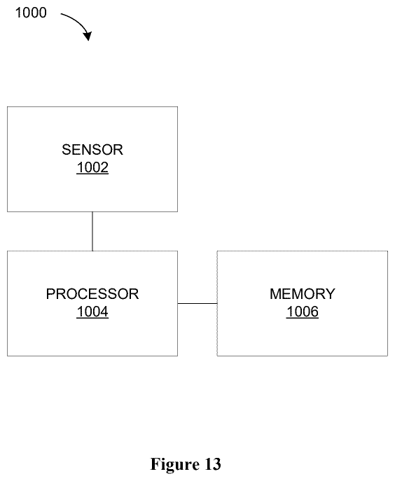

In an aspect, the present disclosure provides for a computing system 1000 as shown in FIG. 13. Various methods in accordance with the present disclosure can be implemented on computing system 1000. Computing system 1000 can include a sensor 1002, a processor 1004, and a memory 1006. In some examples, one or more of the sensor 1002, the processor 1004, and/or the memory 1006 can be integrated into a single device. In some examples, the sensor 1002 may be in electrical communication with the processor 1004. In some examples, the sensor 1002 and the processor 1004 may be in wireless communication. In some examples, the processor 1004 and/or the memory 1006 may be cloud-based, and the sensor 1002 may be capable of communicating with the cloud-based network. The sensor 1002 may include any of the sensor components discussed herein, for example with reference to FIGS. 1-2 or 5-8. The memory 1006 may include instructions for the processor 1004 to execute any of the processes discussed herein, for example any of the processes discussed herein with reference to FIG. 3, 4, or 9. It is to be understood that the computing system 1000 may include one or more of each of the sensor 1002, the processor 1004, and/or the memory 1006. The computing system 1000 may be able to communicate with a user device, for example a personal computing device or a mobile device. In some examples, the computing system 1000 optionally includes a display, which can be used to display parameters calculated from measurements of the sensor 1002.

In some embodiments, software written to perform the methods as described herein is stored in memory in some form of computer readable medium, for example memory, CD-ROM, DVD-ROM, memory stick, flash drive, hard drive, SSD hard drive, server, mainframe storage system and the like.

In some embodiments, the methods of any processor or memory may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PUP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A blood pressure estimation apparatus comprising:
(a) one or more pressure sensors disposed within footwear and configured to measure a time-varying plantar pressure signal from a foot of a wearer, the plantar pressure signal comprising a weight-bearing component and a cardiovascular pulsatile component, wherein the cardiovascular pulsatile component arises from interaction between the wearer's body weight and arterial blood pressure in plantar vasculature;
(b) signal processing circuitry coupled to the one or more pressure sensors and configured to digitize the plantar pressure signal; and
(c) one or more processors configured to:
(i) separate or identify the cardiovascular pulsatile component within the digitized plantar pressure signal,
(ii) determine an amplitude of the cardiovascular pulsatile component as a function of a magnitude of the weight-bearing component, and
(iii) estimate at least one blood pressure parameter from a relationship between the pulsatile amplitude and the weight-bearing pressure magnitude; wherein the apparatus estimates blood pressure without an inflatable cuff and without requiring deliberate user action beyond wearing the footwear.

2. The apparatus of claim 1, wherein the at least one blood pressure parameter comprises mean arterial pressure estimated as the weight-bearing pressure at which the pulsatile amplitude is maximized.

3. The apparatus of claim 2, wherein the one or more processors are further configured to estimate systolic blood pressure and diastolic blood pressure from weight-bearing pressures at which the pulsatile amplitude falls to characteristic fractions of a peak amplitude.

4. The apparatus of claim 1, wherein the relationship between the pulsatile amplitude and the weight-bearing pressure magnitude forms an oscillometric envelope analogous to an oscillometric envelope produced by an inflatable arm cuff instrument.

5. (canceled)

6. (canceled)

7. The apparatus of claim 1, wherein the one or more processors estimate the at least one blood pressure parameter using a machine learning model trained on simultaneously acquired plantar pressure data and reference blood pressure measurements.

8. The apparatus of claim 7, wherein the machine learning model is trained on population-wide data and is fine-tuned for an individual wearer using one or more reference blood pressure measurements.

9. (canceled)

10. (canceled)

11. (canceled)

12. (canceled)

13. The apparatus of claim 1, wherein the footwear comprises a sealed, flexible enclosure filled with a fluid, and wherein at least one of the one or more pressure sensors is mounted within or on the enclosure in fluid communication with the fluid so as to measure pressure reflecting an aggregate plantar pressure applied by the foot.

14. (canceled)

15. The apparatus of claim 13, wherein the fluid provides mechanical low-pass filtering that attenuates high-frequency gait impact artifacts while preserving the cardiovascular pulsatile component.

16. (canceled)

17. The apparatus of claim 13, wherein a single pressure sensor within the sealed enclosure senses an area-averaged plantar pressure over a contact area of the foot by pressure equilibration through the fluid.

18. (canceled)

19. (canceled)

20. The apparatus of claim 1, wherein the one or more processors are further configured to estimate heart rate from a frequency of the cardiovascular pulsatile component.

21. The apparatus of claim 1, wherein the one or more processors are further configured to estimate respiration rate from a modulation of the cardiovascular pulsatile component or of the weight-bearing component.

22. The apparatus of claim 1, further comprising a communication interface configured to receive photoplethysmography (PPG) data from an external wearable device, wherein the one or more processors are configured to:
(a) calculate a blood pressure estimate based at least in part on both a plantar-pressure-based blood pressure estimate and a PPG-derived blood pressure estimate when both the footwear and the external wearable device are worn simultaneously, or
(b) monitor blood pressure, based at least in part on the PPG-derived blood pressure estimate, during periods when the footwear is not worn.

23. The apparatus of claim 22, wherein the plantar-pressure-based blood pressure estimate is used to calibrate or recalibrate a PPG-based blood pressure estimation model running on the external wearable device.

24. The apparatus of claim 1, wherein the one or more processors are configured to receive one or more reference blood pressure measurements from an external instrument and to calibrate the blood pressure estimation based on the reference measurements.

25. (canceled)

26. (canceled)

27. (canceled)

28. (canceled)

29. (canceled)

30. The apparatus of claim 1, further comprising an inertial measurement unit disposed within the footwear, wherein the one or more processors use inertial measurement data to distinguish weight-bearing pressure variations caused by gait from cardiovascular pulsatile variations.

31. The apparatus of claim 1, further comprising one or more pairs of electrical impedance sensing electrodes disposed within the footwear and configured to inject an alternating current into tissue of the wearer's foot and measure a resulting voltage to determine a bioelectrical impedance at one or more excitation frequencies.

32. The apparatus of claim 31, wherein the one or more processors are configured to estimate at least one body water parameter selected from: total body water, extracellular water, intracellular water, and a ratio of extracellular water to intracellular water, from the bioelectrical impedance measured at a plurality of excitation frequencies.

33. The apparatus of claim 32, wherein the one or more processors are configured to detect at least one of: dehydration, edema, fluid retention, or fluid overload, based on deviations of the at least one body water parameter from a baseline value or from a configurable threshold.

34. The apparatus of claim 31, wherein the one or more processors are configured to estimate at least one body composition parameter selected from: fat mass, fat-free mass, lean mass, and skeletal muscle mass, from the bioelectrical impedance measured at a plurality of excitation frequencies.

35. The apparatus of claim 31, wherein the one or more processors are configured to extract an impedance plethysmography waveform from time-varying impedance measurements at a sampling rate sufficient to resolve cardiac-synchronous pulsatile blood volume changes in plantar vasculature of the foot.

36. The apparatus of claim 35, wherein the one or more processors are configured to calculate a blood pressure estimate based at least in part on both the impedance plethysmography waveform and the cardiovascular pulsatile component of the plantar pressure signal.

37.-55. (canceled)
